(19) United States
(12) Patent Application Publication
(10) Pub. No.: US 2023/0255969 A1
(43) Pub. Date: Aug. 17, 2023

(54) TARGETING IMMUNE PATHOLOGIES INDUCED BY HIGHLY PATHOGENIC CORONAVIRUSES

(71) Applicant: National University of Singapore, Singapore (SG)

(72) Inventor: Ashley Lauren St. John, Singapore (SG)

(73) Assignee: National University of Singapore, Singapore (SG)

(21) Appl. No.: 18/006,541

(22) PCT Filed: May 27, 2021
(86) PCT No.: PCT/SG2021/050295

§ 371 Date: Jan. 23, 2023

(30) Foreign Application Priority Data
Jul. 23, 2020 (SG) 10202007071Q

(51) Int. Cl. A61K 31/517; A61K 31/352; A61K 31/4535; A61K 31/155; A61K 31/427; A61P 31/14

(52) U.S. Cl. CPC A61K 31/517; A61K 31/352; A61K 31/4535; A61K 31/155; A61K 31/427; A61P 31/14

(57) ABSTRACT

The present invention relates to treatment of coronavirus-induced disease, specifically COVID-19 disease, wherein the disease is characterized by mast cell degranulation, acute inflammation, pulmonary and/or vascular pathologies. The treatment comprises administering to a subject a composition comprising a mast cell stabilizer and/or inhibitor of mast cell products, such as TY-51469, nafamostat mesylate, cromolyn, or ketotifen. The invention also provides a method of diagnosing a subject as having SARS-CoV-2, the method comprising determining the level and/or activity of a mast cell protease such as chymase or tryptase in the serum from a subject.

### FIELD OF THE INVENTION

The present invention relates to treatment of coronavirus-induced disease, wherein the disease is characterized by mast cell degranulation, acute inflammation, pulmonary and/or vascular pathologies. More particularly, the invention relates to methods of treating immune pathologies associated with COVID-19 disease by inhibiting mast cell activation and/or by inhibiting proteases, such as chymase and tryptase. The invention also provides methods of diagnosing, monitoring and treating a subject as having a coronavirus-induced disease.

### BACKGROUND

COVID-19 is caused by the SARS-CoV-2 virus, a highly pathogenic coronavirus, which emerged as a novel infectious disease at the end of 2019. Subsequently, in 2020, the virus and infection it causes were named and declared a pandemic by the World Health Organization [Organization, W. H. WHO Timeline —COVID-19. June 2020; worldwidewebdotwhodotint/news-room/detail/27-04-2020-who-timeline---covid-19]. Although we are still learning about the clinical signs and symptoms of this infection, initial reports describe a highly varied clinical disease presentation that is unique compared to other viral infectious diseases. To date, there are no effective targeted intervention strategies to treat or prevent COVID-19 disease. In humans, SARS-CoV-2 induces a respiratory illness that can be life-threatening, which coincides with systemic changes to the immune and coagulation systems. Some of the key signs of disease described to date in the first clinical reports include rash, coagulopathy, and acute respiratory distress syndrome (ARDS) [Guan, W. J. et al., Eur Respir J 55: 2000547 (2020)]. Severe disease is also characterized by tissue and organ damage and a heightened risk of shock. Less frequent presentations include neurological complications, including encephalitis and thrombotic complications resulting in stroke, which could also potentially result from the altered coagulation and vascular homeostasis [St John, A. L. & Rathore, A. P. S. J Immunol 205: 555-564 (2020)].

COVID-19 is a new infectious disease so there are no targeted treatments/therapeutics aside from supportive care, potentially involving admission to an intensive care unit. Most current strategies that have been raised in the literature, but which aren't approved, involve targeting of the virus replication cycle using antiviral drugs. For example, remdesivir was given emergency use authorization in severe COVID-19 patients based on early promising results in clinical trials [worldwidewebdotfdadotgov/emergency-preparedness-and-response/mcm-legal-regulatory-and-policy-framework/emergency-use-authorization #covidtherapeutics (2020); worldwidewebdotniaiddotnihdotgov/news-events/nih-clinical-trial-shows-remdesivir-accelerates-recovery-advanced-covid-19 (2020)]. However, it should be noted that remdesivir was recommended for early termination from an Ebola clinical trial due to safety monitoring concerns and lack of efficacy when compared to other treatment groups [Mulangu, S. et al., N Engl J Med 381: 2293-2303 (2019)]. Remdesivir is a nucleoside analogue that targets replication of RNA viruses, so its proposed theoretical mechanism of action against Ebola and SARS-CoV-2 is the same [Eastman, R. T. et al., ACS Cent Sci 6: 672-683 (2020)] and there is a strong possibility that similar obstacles to clinical development as for Ebola virus disease will be present for COVID-19.

To date, there are several vaccines that have been given emergency use authorizations for COVID-19 prevention and emergency use therapeutic options are available for its treatment in the most severe patients. Alternative proposed strategies to treat the virus itself or the disease that it induces involve the use of host-cytokine targeting or virus-targeting monoclonal antibodies [Pinto, D. et al., Nature 583: 290-295 (2020); Wang, C. et al., Nat Commun 11: 2251 (2020)]. Some humanized monoclonal antibodies against the spike protein have been made and tested in cell culture [Pinto, D. et al., Nature 583: 290-295 (2020); Wang, C. et al., Nat Commun 11: 2251 (2020)], but it is not known whether these will work in vivo in animal models and humans, particularly since humans have mostly already cleared the virus itself at time points when severe disease occurs [St John, A. L. & Rathore, A. P. S. J Immunol 205: 555-564 (2020)]. There is also evidence that cytokines are elevated during COVID-19 disease, which has led to speculation that a cytokine storm may be the cause of severe disease during COVID-19 [St John, A. L. & Rathore, A. P. S. J Immunol 205: 555-564 (2020)]. Although not yet experimentally tested, it raised the possibility of targeting cytokines to limit COVID-19 induced lung pathology, with much emphasis to date on IL-6. Although the efficacy of IL-6 blockade in preventing ARDS is not yet known, early data suggesting that suppressing it may be associated with increased secondary infections in COVID-19 patients supports the importance of proceeding with caution when suppressing cytokine responses [Kimmig, L. M. et al., medRxiv, 2020.2005.2015.20103531 (2020)], so the utility of this method is not known.

There are obstacles to the production and distribution of COVID-19 vaccines to meet the world-wide need and the long-term efficacy of these vaccines is unknown [worldwidewwebdotwhodotint/blueprint/priority-diseases/key-action/novel-coronavirus-landscape-ncov.pdf. (2020); Thanh Le, T. et al., Nat Rev Drug Discov 19: 305-306 (2020); Corey, B. L., Mascola, J. R., Fauci, A. S. & Collins, F. S. Science 368: 948-950 (2020)]. Indeed, even with best estimates, COVID-19 is expected to cause a sustained burden in humans for many years. Based on the emergence of SARS, MERS and now COVID-19 as diseases caused by coronaviruses in the past 20 years alone, there is also risk that new coronaviruses may emerge for which vaccines may be not yet developed but for which the targeting strategy described herein could be effective, given that it is a host-directed rather than virus-directed therapeutic strategy.

There is a need to provide treatments that alleviate one or more coronavirus-induced inflammation, pulmonary and vascular pathologies.

### SUMMARY OF THE INVENTION

In the context of other viral infections and during sterile inflammation, some of the signs and symptoms of coronavirus infection are consistent with the effects of mast cell activation. Mast cells (MCs) undergo a process called degranulation, where they release specialized pre-formed mediators that can regulate inflammation in tissues and modulate the vasculature (FIG. 1). For example, our previous work showed that during dengue infection the mast cell-derived mediator tryptase can promote vascular leakage and mast cell-derived serotonin can promote thrombocytopenia [Rathore, A. P. et al., J Clin Invest 130: 4180-4193 (2019); Masri, M. F. B., Mantri, C. K., Rathore, A. P. S. & John, A. L. S. Blood 133: 2325-2337 (2019)], while mast cell chymase promotes blood brain barrier leakage during Japanese encephalitis infection [Hsieh, J. T., Rathore, A. P. S., Soundarajan, G. & St John, A. L. Nat Commun 10: 706 (2019)]. Other triggers that activate mast cells can also influence blood pressure through the angiotensin-converting enzyme chymase [Tchougounova, E., Pejler, G. & Abrink, M. J Exp Med 198: 423-431 (2003)], which we have also observed is elevated during certain viral infections and possibly contributes to vascular permeability and edema [Rathore, A. P. et al., J Clin Invest 130: 4180-4193 (2019); Tissera, H. et al., J Infect Dis 216: 1112-1121 (2017); St John, A. L., Rathore, A. P., Raghavan, B., Ng, M. L. & Abraham, S. N. Elife 2: e00481 (2013)]. Mast cells products also promote other pathogenic sequalae.

Although it has not yet been reported in the literature that mast cells are activated by SARS-CoV-2 infection, we hypothesized that they may be since they are located in the lungs and lining the blood vessels where they can contribute to pulmonary and vascular pathologies. Moreover, there are similarities between the signs and symptoms of COVID-19 disease and mast cell activation in other contexts, including lung tissue damage, coagulopathy, damage to blood vessels, rash, and increased incidence of encephalitis. Thus, we propose that targeting mast cells or their products could ameliorate the immune pathologies that are associated with COVID-19 vascular and pulmonary disease.

Targeting mast cells to prevent/treat COVID-19 disease, if successful, could take several forms, including the use of mast cell stabilizers to block the release of mast cell granules; use of protease inhibitors to prevent the activity of proteases such as chymase and tryptase on downstream processes they regulate (such as activation of the vascular endothelium, influence on the renin-angiotensin system, regulation of blood pressure, influence on pulmonary edema and pulmonary hypertension, activation of matrix metalloproteinases, permeabilization of the blood brain barrier, fibrin deposition, etc.); use of specific receptor blockers to prevent the activity of mast cell derived biogenic amines (such as serotonin influence on coagulation); and inhibitors of mast cell derived lipid products that regulate inflammation and vasoactivation (FIG. 2). We also anticipate that these inhibitors could be used in combination, for example to prevent the activation of mast cells, while also inhibiting the products they have already released, or to prevent the combined activities of proteases and other classes of mast cell derived products such as biogenic amines or lipids which work together to induce coagulation. It is possible that some of these drugs could influence the virus directly, but that is not our intended use, since the viral titers appear to be declining in the airways at the time points when severe disease occurs [St John, A. L. & Rathore, A. P. S. J Immunol 205: 555-564 (2020)]. This suggests that strategies to improve outcomes involving immune pathology could be more productive in treating and preventing severe disease than antivirals.

According to a first aspect, the present invention provides a composition comprising a mast cell stabilizer and/or inhibitor of mast cell products for prophylaxis or treatment of coronavirus-induced disease, wherein the disease is characterized by mast cell degranulation, acute inflammation, pulmonary pathology and/or vascular pathology.

In some embodiments the coronavirus is selected from the group comprising Severe Acute Respiratory Syndrome-associated coronavirus (SARS-CoV), SARS-Cov-2 and Middle East Respiratory Syndrome-associated coronavirus (MERS-CoV).

In some embodiments the mast cell stabilizer is selected from one or more of a group comprising cromolyn, nedocromil, pemirolast, lodoxamide, tranilast, glucosamine, N-acetylglucosamine, FPL 52694, aloe vera, quercetin, chondroitin sulfate, dehydroleucodine, mast cell stabilizer TF002, rupatadine, loratadine, cetirizine, clemastime, fexofenadine, diphenhydramine, chlorpheniramine, azelastine, olopatadine, naphazoline, ketotifen, emedastine, ebrotidine, calcium channel blocker, a cytochrome P450 inhibitor, a histamine antagonist, and the inhibitor of mast cell products is selected from one or more of a group comprising zafirlukast, ketanserin, montelukast, pranlukast, zileuton, SM-12502, rupatadine, PAF-targeting antibodies, xanthine derivatives, methylxanthines like theophylline oxtriphylline, dyphylline, aminophylline, bupropion, curcumin, catechins, aprotinin, serpin, a chymase inhibitor, TY-51469, chymostatin, leupeptin, APC-336, SUN-C8257, NK3201, R0566852, BCEAB, NK3201, TEI-E548, APC-2095, RWJ-355871, TPC-806, ZIGPFM, AAPF-S-Bzl, Bowman-Birk soybean protease inhibitor, BI-1942, TEI-f00806, BAY-1142524, fulacimstat, ASB17061, Polygonum, SFTI-1 and derivatives, bevacizumab, ranibizumab, lapatinib, sunitinib sorafenib, axitinib, pazopanib, thiazolidinediones, benzoxazole, benzthiazole, benzinidzole, CP105,696, laropiprant, acetylsalicylic acid (ASA), indomethacin, sodium meclofenamate (FEN), phenylbutazone (PB), phloretin phosphates (PP), SC-19220, diethylcarbamazine citrate (DECC), protamine and polybrene, a tryptase inhibitor, nafamostat mesylate, BMS-262084, BMS-363131, BSM-36130, Guanadino β-lactams that inhibit tryptase, delta inhibitors of tryptase, benzamidine dimers that inhibit tryptase, piperidine containing 4-carboxy azetidinone tryptase inhibitors, APC-2059, BAY 443428, phenylglycylcarbonyl benzylamines, Peptidyl heterocyclic ketones, Guanidino Bicyclic lactam, Amino or Amidino dimers, Peptidomimetic inhibitors, MOL-6131, RWJ-56423, RWJ-58643, RWJ-51084, BABIM (bis-(5-amidino-2-benzimidazoyl) methane, APD-8, AMG-126737, 4-chlorobenzyoyl ester of 4-hydroxytetronic acid and its p-toluate tetronic acid derivatives, M-58538, AY-0068, PMD-3027, Cyclotheonamide E4 and E5, and amidinobenzofuran derivatives.

In some embodiments the composition comprises one or more mast cell stabilizers and/or inhibitor of mast cell products selected from the group comprising ketotifen [IUPAC Name: 2-(1-methylpiperidin-4-ylidene)-6-thiatricyclo[8.4.0.03,7]tetradeca-1(14),3(7),4,10,12-pentaen-8-one], cromolyn [IUPAC Name: 5-[3-(2-carboxy-4-oxochromen-5-yl)oxy-2-hydroxypropoxy]-4-oxochromene-2-carboxylic acid], nafamostat mesylate [IUPAC Name: (6-carbamimidoylnaphthalen-2-yl) 4-(diaminomethylideneamino)benzoate; methanesulfonic acid], TY-51469 [IUPAC Name: 2-[4-[(5-fluoro-3-methyl-1-benzothiophen-2-yl)sulfonylamino]-3-methylsulfonylphenyl]-1,3-thiazole-4-carboxylic acid] and ketanserin [IUPAC Name: 3-[2-[4-(4-fluorobenzoyl)piperidin-1-yl]ethyl]-1H-quinazoline-2,4-dione].

In some embodiments;
cromolyn is formulated for administration to adults at 200 mg 4× a day, and children at 100 mg 4× a day, (but higher doses may be needed);
ketotifen is formulated for administration orally at 1 mg per day in adults or 0.110 mg per pound of body weight including for children (higher doses may be needed);
nafamostat mesylate is formulated for administration at 20-50 mg infusion, or 2-10 mg/kg per day (higher doses may be needed);
TY-51469 is formulated for administration at 0.1-100 mg/kg per day (higher doses may be needed); and/or
Ketanserin is formulated for administration at 10-100 mg twice a day (higher doses may be needed).

According to another aspect, the present invention provides a method of diagnosing a subject as having a coronavirus-induced disease, wherein the disease is characterized by mast cell degranulation, acute inflammation, pulmonary pathology, vascular pathology, the method comprising:
(a) obtaining a biological sample from the subject;
(b) determining the level and/or activity of at least one biomarker in the biological sample from the subject;
(c) comparing the level and/or activity of the at least one biomarker in the biological sample to a reference level and/or activity of the at least one biomarker;
(d) identifying the subject as having the disease or having an increased risk of developing the disease if the level and/or activity of the at least one biomarker is greater than the reference level and/or activity of the at least one biomarker;
(e) optionally preventing or treating the disease by administering an efficacious amount of a composition of any aspect of the invention.

In some embodiments, the control sample is a sample from a healthy patient, a patient having a mild form of the coronavirus-induced disease, or a patient having a severe form of the coronavirus-induced disease.

In some embodiments, the biomarker is a mast cell protease, preferably selected from the group comprising chymase and tryptase.

In some embodiments:
a) a mast cell protease level and/or activity greater than 1 standard deviation above the mean for patients during acute disease or during disease resolution indicates the subject should be monitored for severe disease and/or complications; or
b) a mast cell protease level above a normal level of about 300 pg/mL for tryptase or 3 ng/mL for chymase indicates the subject should be monitored for severe disease.

According to another aspect, the present invention provides a method of prophylaxis or treatment of a coronavirus-induced disease, wherein the disease is characterized by mast cell degranulation, acute inflammation, pulmonary pathology, vascular pathology, comprising administering to a subject in need thereof an efficacious amount of a composition of any aspect of the invention.

In some embodiments, the coronavirus is selected from the group comprising SARS-CoV, SARS-CoV-2 and MERS-CoV.

In some embodiments, the subject is administered:
cromolyn at 200 mg 4× a day (adults), or at 100 mg 4× a day (children), (higher doses may be needed);
ketotifen at 1 mg per day (adults) or at 0.110 mg per pound of body weight (adults or children), (higher doses may be needed);
nafamostat at 20-50 mg by infusion, or 2-10 mg/kg per day, (higher doses may be needed);
TY-51469 at 0.1-100 mg/kg per day, (higher doses may be needed); and/or
Ketanserin at 10-100 mg twice a day, (higher doses may be needed).

According to another aspect, the present invention provides a method of monitoring the efficacy of the method of prophylaxis or treatment of any aspect of the invention, comprising serially measuring the level and/or activity of a mast cell protease, preferably selected from the group comprising chymase and tryptase, in at least one sample from said subject.

According to another aspect, the present invention provides use of a composition of any aspect of the invention for the manufacture of a medicament for the prophylaxis or treatment of a coronavirus-induced disease, wherein the disease is characterized by mast cell degranulation, acute inflammation, pulmonary pathology, vascular pathology.

### BRIEF DESCRIPTION OF THE FIGURES

FIG. 1 shows schematically a hypothesis for mechanisms of MC regulation of immune pathology and coagulopathy during COVID-19. MCs can respond directly to some viruses and degranulate. In the presence of antibodies such as IgG or IgE the magnitude of MC degranulation increases. Upon degranulation and activation, release of MC products can induce (1) coagulopathy by serotonin-mediated platelet activation, (2) microvascular permeability by shedding endothelial glycocalyx, which can be exacerbated by the actions of MC products including chymase on hypertension, and (3) vascular leakage by breakdown of endothelial tight junctions. Tryptase is a MC-derived protease that can degrade both glycocalyx and tight junctions between endothelial cells. (4) While tryptase has a known prominent role in peripheral tissues, chymase was identified as inducing blood brain barrier (BBB) permeability and facilitating viral neuroinvasion during Japanese encephalitis virus infection. Since tryptase and chymase have different substrate specificities and may be released differentially depending on virus tissue tropism, they may play unique roles at differing tissue sites. Many of these mechanisms (1-4) have been shown using other RNA-viruses including dengue and Japanese encephalitis viruses but may be relevant for SARS-CoV-2. (5) MCs are also located in the lungs where they regulate the tissue during inflammatory conditions. MC activation during COVID-19 disease might induce the pathological influences that are observed during other conditions that activate lung MCs, such as asthma. Image was created with biorender.com.

FIG. 2 shows a schematic of potential MC-targeting therapeutics for SARS-CoV-2 and their mechanisms of action.

FIG. 3 shows an experimental design for coronavirus therapeutics testing. The hACE2-ki mouse model, where the human ACE2 enzyme is knocked-in is used in order to allow for more robust treatment and testing of drug candidates, although alternatives may be substituted if better models are developed. (1) Mice are inoculated intra-nasally with SARS-CoV-2, SARS-CoV, or MERS-CoV to achieve a respiratory tract infection analogous to the natural route in humans. (2) There are 3 testing groups: (i) mice are pre-treated to determine if there is a potential to prevent disease, (ii) mice are treated daily following infection to determine the therapeutic potential of a candidate drug, and (iii) a delayed treatment group where mice are given injections several days after infection. Drugs are provided daily and any that are effective are tested in combinations. (3) Readouts of experimental testing are related to reversing the inflammation and severe vascular pathologies and therefore the following are assessed and recorded: tissue damage (especially lung damage) by histology, coagulation biomarkers and cellular read outs, inflammatory biomarkers and cellular infiltration in the lung tissue, and the survival and/or clinical score. An effective drug(s) will improve one or more of these readouts when compared to untreated or vehicle treated controls. Image was created with biorenderdotcom.

FIG. 4 shows a mouse model and quantification of SARS-CoV-2 infection in mice. (A) C57BL/6 mice were inoculated intranasally with AAV9-hACE2—to induce hACE2 expression in the airways. SARS-CoV-2 (2×10⁷ TCID₅₀) was inoculated intranasally into AAV9-hACE2 C57BL/6 mice. Blood was taken on days 1, 3, 5, and 7, and organs were harvested after 5 or 7 days for histology and virus quantification. (B) Virus quantification from the organs harvested shows detection in the lung, spleen, liver, kidney, brain, and bone marrow both Days 5 and 7.

FIG. 5 shows histological images of degranulation of Mast Cells (MCs) in SARS-CoV-2 infected mice. Histology images of toluidine blue-stained trachea sections from (A) uninfected and (B) SARS-CoV-2 infected mice. In uninfected tissues in panel A, arrows indicate MCs that could be identified by toluidine blue, all of which were resting and not degranulating. In panel B, degranulating MCs) could be observed in SARS-CoV-2 infected mice (indicated by arrows) as well as tissue edema and airway narrowing.

FIG. 6 shows immunoblot of systemic elevation of MC-derived proteases in SARS-CoV-2 infection. Western blot images after chymase detection in serum Days 3, 5 and 7 post-infection shows systemic elevation of chymase, which was quantitated by densitometry from 3 individual mouse samples and presented as fold-increase over uninfected controls. Error bars represent the SEM. Chymase was significantly elevated in serum of infected mice compared to uninfected controls, determined by 1-way ANOVA with Dunnett's post-test; p<0.05, p<0.01.

FIG. 7 shows experiment regimen and resulting lung pathology of Cynomolgus macaques infected with SARS-CoV-2. (A) Cynomolgus macaques were infected intratracheally with SARS-CoV-2 and monitored for 21 days prior to necropsy. (B) Abnormal findings related to lung tissue observed at the time of necropsy were recorded and effected all animals. (C) Images of non-human primate (NHP) lungs at the time of necropsy show areas of hemorrhaging and necrotic spots on the lung surface. Boxed region is enlarged. (D) To confirm infection, viral detection was performed by PCR at regular intervals post-infection using swabs from multiple mucosal tissues, lung lavage, and nasal rinses. All NHPs were positive for SARS-CoV-2 infection multiple days after inoculation.

FIG. 8 shows photomicrographs of histological detection of activation of MCs coinciding with lung pathology in Cynomolgus macaques. (A) Histological assessment of lung tissues by H&E staining shows hemorrhaging of the tissue and free RBCs within the lung alveolar spaces. (B) Inset corresponding to the boxed region of panel A. (C) Some RBCs in the tissue proximal to a blood vessel are indicated by arrows and cellular infiltrates are circled. (D) Multiple examples of degranulating or hypogranulated MCs are provided, observed in toluidine blue stained lung tissue sections. The MCs are enlarged in the boxed insets. MCs from various regions of the lungs are presented, including i. lower lung tissue, ii. & iii. Trachea, iv. Lung tissue proximal to a bronchiole, and v. & vi. Lung tissue with hemorrhaging. For (E-G), lung sections were stained for MC heparin to indicate the location of MC granules and DAPI to identify cellular nuclei and tissue structures. MCs are indicated with white arrows. (E) MCs were observed degranulating in the lung of SARS-CoV-2 infected primates in sections of a biopsy of lung tissue that did not have overt hemorrhaging visible on the lung surface at necropsy. (F) MCs appear more densely packed in the lung biopsy from a hemorrhagic lobe of the lung and again, degranulation is observed based on staining for MC-heparin. (G) Images of degranulating MCs are presented at higher magnification.

FIG. 9 shows transcriptional signatures of MC associated genes with severe COVID-19. Genes associated with a (A-B) MC-specific or (C-D) MC/basophil phenotype that were significantly regulated in severe COVID-19 patients. Heatmap shows the LSmean expression values of MC-specific or MC/basophil phenotype genes in severe COVID-19 patients (n=6) at the various days relative to the peak severity with respect to respiratory function (day 0). Clusters of genes that were significantly upregulated during the acute phase (A, C) or resolution phase (B, D) are presented. (E) Normalized expression levels of the MC-specific genes shown in A and C over time, in the severe COVID-19 patients. (F) Pathway analysis indicates a significant perturbation of pathways associated with MC function and/or MC-precursor maturation.

FIG. 10 shows a bar graph of serum chymase levels in patients. Levels of chymase in the serum of acute COVID-19 patients recruited in Singapore were compared to the concentrations previously detected and reported in a study of acute dengue patients [St John, A. L., et al., Elife 2: e00481 (2013)], and to healthy controls. Concentrations were compared by 1-way ANOVA with Bonferroni's post-test to determine p-values. N=10 for controls, N=108 for dengue and N=3 for COVID-19. For ** p<0.01; for***, p<0.001.

FIG. 11 shows a bar graph of percentage MC degranulation. Mast cell degranulation in response to SARS-CoV-2 Spike protein was reduced by treatment with the mast cell stabilizing drug cromolyn. Purified recombinant Spike protein from SARS-CoV-2 was conjugated to beads to approximate the size of virus particles. Significant and dose-dependent mast cell degranulation was induced by Spike-coated beads, but not in mast cells treated with the mast cell stabilizing drug cromolyn.

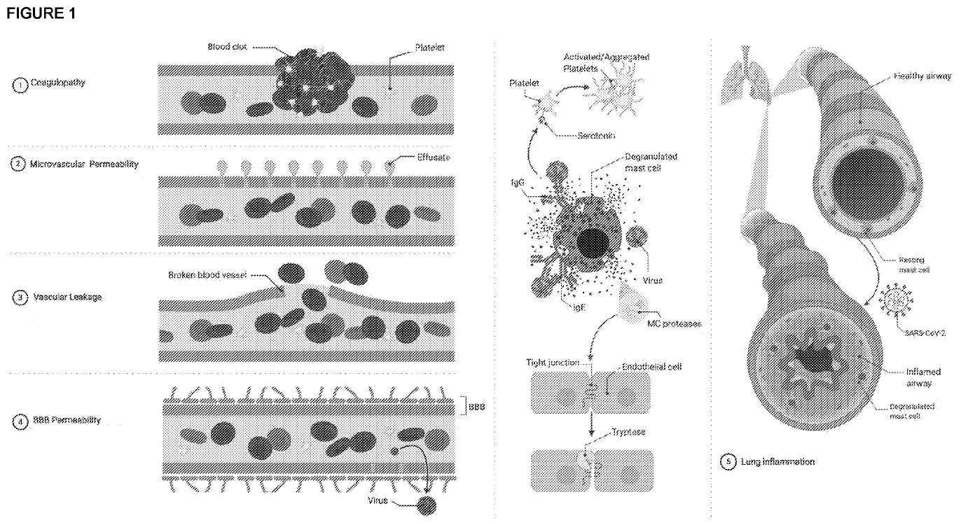

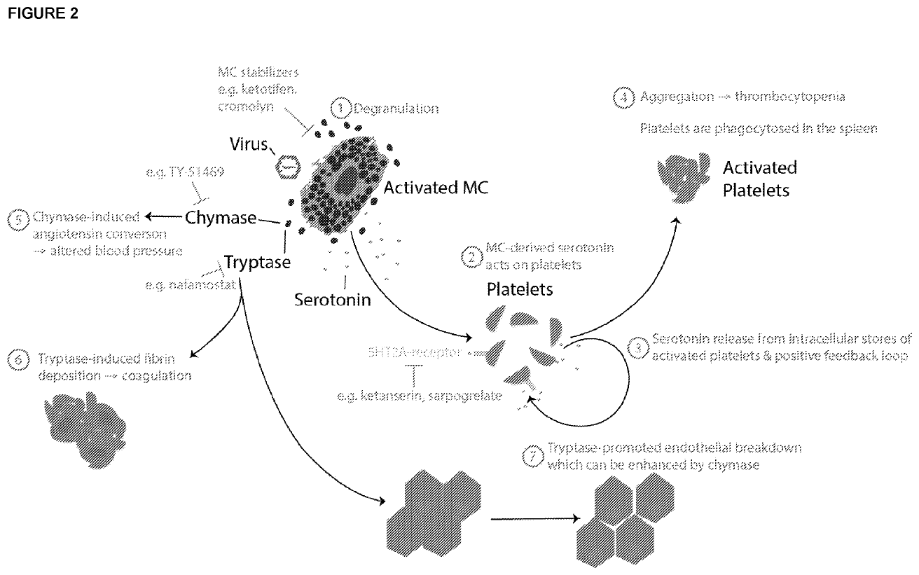

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

When mast cells are activated in vivo, their products can be used as biomarkers of inflammation and tissue damage. Mast cell proteases may be effective biomarkers individually or in combination to prognosticate severe coronavirus disease and, specifically, COVID-19. Chymase and tryptase are mast cell products that have differing half-lives, and we expect they will be present in the serum at high concentrations at the time of hospital admission or when seeking medical attention, beginning approximately 4-7 days after infection. At the time of resolution of infection, the levels of chymase and tryptase should return to normal, approximately 7-14 days after infection, However, if high levels of proteases remain during the resolution stage of disease it is an indication of severe disease requiring hospitalization or more urgent attention, or therapeutic intervention. High levels of tryptase, specifically, would indicate a heightened risk of developing shock. Knowing that approximately ⅓ of COVID-19 patients experience more severe disease, patients with chymase or tryptase (or other MC-derived protease) levels greater than 1 standard deviation above the mean for COVID-19 patients during acute disease or during disease resolution should be monitored for severe disease or COVID-19 complications. In most cases, serum samples would be used, but plasma, urine, saliva or whole blood samples could be used. Alternatively, patients with protease levels outside of the normal healthy rage, (above approximately 300 pg/mL for tryptase or 3 ng/mL for chymase) could be monitored for severe disease. Also, rather than measuring their concentrations, their enzymatic activities could be measured. The efficacy of therapeutic intervention can also be monitored by serially measuring chymase and/or tryptase levels.

The mast cell modulator may comprise a mast cell stabilizer, wherein the mast cell stabilizer comprises a calcium channel blocker, a cytochrome P450 inhibitor or a histamine antagonist. The mast cell modulator may comprise at least one cromolyn, nedocromil, pemirolast, lodoxamide, tranilast, glucosamine, N-acetylglucosamine, FPL 52694, aloe vera, quercetin, chondroitin sulfate, dehydroleucodine, mast cell stabilizer TF002, rupatadine, loratadine, cetirizine, clemastime, fexofenadine, diphenhydramine, chlorpheniramine, azelastine, olopatadine, naphazoline, ketotifen, emedastine, and ebrotidine, combinations thereof and pharmaceutical compositions thereof. The mast cell modulator may inhibit mast cell-derived products in the subject. The mast cell modulator may comprise a platelet activating factor inhibitor, a protease inhibitor, a VEGF inhibitor, a prostaglandin inhibitor, or a heparin inhibitor. The mast cell modulator may comprise at least one of zafirlukast, montelukast, pranlukast, zileuton, SM-12502, rupatadine, PAF-targeting antibodies, xanthine derivatives, methylxanthines like theophylline oxtriphylline, dyphylline, aminophylline, bupropion, curcumin, catechins, aprotinin, serpin, a chymase inhibitor, TY-51469, chymostatin, leupeptin, APC-336, SUN-C8257, NK3201, R0566852, BCEAB, NK3201, TEI-E548, APC-2095, RWJ-355871, TPC-806, ZIGPFM, AAPF-S-Bzl, Bowman-Birk soybean protease inhibitor, BI-1942, TEI-f00806, BAY-1142524, fulacimstat, ASB17061, Polygonum, SFTI-1 and derivatives, bevacizumab, ranibizumab, lapatinib, sunitinib sorafenib, axitinib, pazopanib, thiazolidinediones, benzoxazole, benzthiazole, benzinidzole, CP105,696, laropiprant, acetylsalicylic acid (ASA), indomethacin, sodium meclofenamate (FEN), phenylbutazone (PB), phloretin phosphates (PP), SC-19220, diethylcarbamazine citrate (DECC), protamine and polybrene, a tryptase inhibitor, nafamostat mesylate, BMS-262084, BMS-363131, BSM-36130, Guana-dino β-lactams that inhibit tryptase, delta inhibitors of tryptase, benzamidine dimers that inhibit tryptase, piperidine containing 4-carboxy azetidinone tryptase inhibitors, APC-2059, BAY 443428, phenylglycylcarbonyl benzylamines, Peptidyl heterocyclic ketones, Guanidino Bicyclic lactam, Amino or Amidino dimers, Peptidomimetic inhibitors, MOL-6131, RWJ-56423, RWJ-58643, RWJ-51084, BABIM (bis-(5-amidino-2-benzimidazoyl) methane, APD-8, AMG-126737, 4-chlorobenzyoyl ester of 4-hydroxytetronic acid and its p-toluate tetronic acid derivatives, M-58538, AY-0068, PMD-3027, Cyclotheonamide E4 and E5, amidinobenzofuran derivatives, combinations thereof and pharmaceutical compositions thereof.

A mast cell stabilizer or compound inhibiting mast cell products or combination of therapeutics targeting mast cells could be provided at the time of presentation to the clinic to prevent mast cell induced immune pathology in the lung and mast cell-induced coagulopathy. Key classes of drugs to be used individually or in combination include the mast cell stabilizers, ketotifen and cromolyn; the protease inhibitors nafamostat mesylate (specifically used at concentrations to target the enzyme tryptase [Rathore, A. P. et al., J Clin Invest 130: 4180-4193 (2019)], rather than high non-specific concentrations that could be antiviral through off-target effects as was indicated by a recent in vitro study that did not involve mast cells [Wang, M. et al., Cell Res 30: 269-271 (2020)]), and TY-51469, serotonin receptor blocking agents (e.g. ketanserin), and inhibitors of the leukotriene and prostaglandin pathways. Key compounds of specific interest include cromolyn, ketotifen, nafamostat mesylate, TY-51469 and Ketanserin.

Alternatively, a mast cell stabilizer or inhibitor of MC products could be provided to severe patients. For example, nafamostat mesylate could be provided to patients in the ICU to reverse the vascular coagulation resulting from MC tryptase at late disease time points.

### Definitions

Certain terms employed in the specification, examples and appended claims are collected here for convenience.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “mast cell stabilizer or inhibitor of MC products” is to be broadly interpreted as a compound that is any chemical that modifies the effects of mast cells in the body by inhibiting mast cell degranulation or the products produced by degranulation. More particularly, the term encompasses compounds that inhibit the effect of mast cell activation on acute inflammation, pulmonary pathology and vascular pathology following coronavirus infection. Mast cell stabilizer compounds according to the invention may be selected from one or more of a group comprising cromolyn, nedocromil, pemirolast, lodoxamide, tranilast, glucosamine, N-acetylglucosamine, FPL 52694, aloe vera, quercetin, chondroitin sulfate, dehydroleucodine, mast cell stabilizer TF002, rupatadine, loratadine, cetirizine, clemastime, fexofenadine, diphenhydramine, chlorpheniramine, azelastine, olopatadine, naphazoline, ketotifen, emedastine, ebrotidine, calcium channel blocker, a cytochrome P450 inhibitor, a histamine antagonist, and the inhibitor of mast cell products is selected from one or more of a group comprising zafirlukast, ketanserin, montelukast, pranlukast, zileuton, SM-12502, rupatadine, PAF-targeting antibodies, xanthine derivatives, methylxanthines like theophylline oxtriphylline, dyphylline, aminophylline, bupropion, curcumin, catechins, aprotinin, serpin, a chymase inhibitor, TY-51469, chymostatin, leupeptin, APC-336, SUN-C8257, NK3201, R0566852, BCEAB, NK3201, TEI-E548, APC-2095, RWJ-355871, TPC-806, ZIGPFM, AAPF-S-Bzl, Bowman-Birk soybean protease inhibitor, BI-1942, TEI-f00806, BAY-1142524, fulacimstat, ASB17061, Polygonum, SFTI-1 and derivatives, bevacizumab, ranibizumab, lapatinib, sunitinib sorafenib, axitinib, pazopanib, thiazolidinediones, benzoxazole, benzthiazole, benzinidzole, CP105,696, laropiprant, acetylsalicylic acid (ASA), indomethacin, sodium meclofenamate (FEN), phenylbutazone (PB), phloretin phosphates (PP), SC-19220, diethylcarbamazine citrate (DECC), protamine and polybrene, a tryptase inhibitor, nafamostat mesylate, BMS-262084, BMS-363131, BSM-36130, Guana-dino β-lactams that inhibit tryptase, delta inhibitors of tryptase, benzamidine dimers that inhibit tryptase, piperidine containing 4-carboxy azetidinone tryptase inhibitors, APC-2059, BAY 443428, phenylglycylcarbonyl benzylamines, Peptidyl heterocyclic ketones, Guanidino Bicyclic lactam, Amino or Amidino dimers, Peptidomimetic inhibitors, MOL-6131, RWJ-56423, RWJ-58643, RWJ-51084, BABIM (bis-(5-amidino-2-benzimidazoyl) methane, APD-8, AMG-126737, 4-chlorobenzyoyl ester of 4-hydroxytetronic acid and its p-toluate tetronic acid derivatives, M-58538, AY-0068, PMD-3027, Cyclotheonamide E4 and E5, and amidinobenzofuran derivatives.

References herein (in any aspect or embodiment of the invention) to said mast cell stabilizer compounds includes references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a stabilizer compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a serotonergic compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1 S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g.(+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by mast cell stabilizer compounds are any solvates of the compounds and their salts. Preferred solvates are solvates formed by the incorporation into the solid-state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallizing the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

The term “antagonist”, or “inhibitor” as it is used herein, refers to a molecule that decreases the amount or the duration of the effect of mast cell degranulation, thereby inhibiting acute inflammation, pulmonary pathology and vascular pathology following coronavirus infection.

Compounds of the present invention will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of a compound in any pharmaceutical formulation used in accordance with the present invention will depend on various factors, such as the severity of the condition to be treated, the particular patient to be treated, as well as the compound(s) which is/are employed. In any event, the amount of a compound in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated.

For example:
cromolyn may be used in adults at 200 mg 4× a day, and children 100 mg 4× a day, but higher doses may be needed;
ketotifen may be used orally 1 mg per day in adults or 0.110 mg per pound of body weight including for children, although higher doses may be needed;
nafamostat mesylate may be administered at 20-50 mg infusion, or 2-10 mg/kg per day;
TY-51469 may be administered at 0.1 to 100 mg/kg per day;
Ketanserin may be administered at 10-100 mg twice a day.

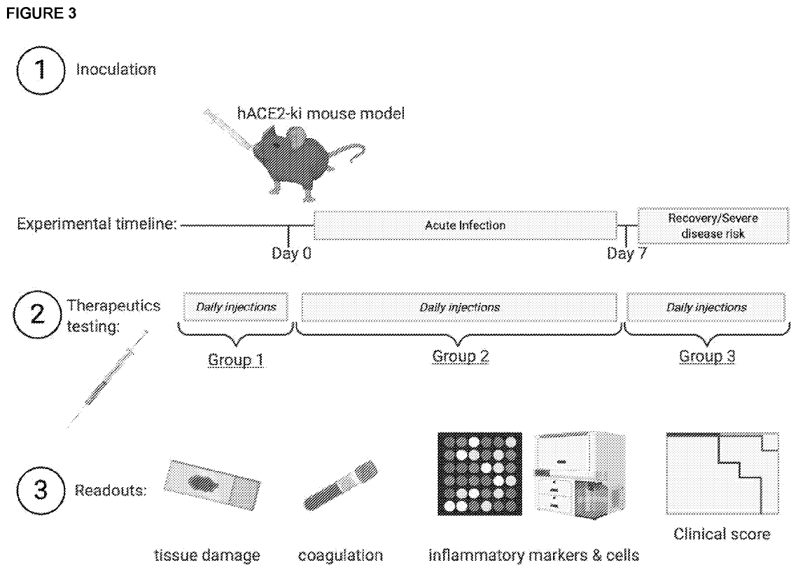

COVID-19 remains unique compared to other viral infectious diseases that activate MCs. For example, it infects the lung tissue directly, in contrast to viruses such as DENV and JEV that have been reported to activate MCs, but do not cause direct infection of the lung tissue. Even compared to closely related viruses, MERS and SARS, there are key differences in the course of disease and clinical manifestations. Specifically, the association with altered coagulation is a unique factor of COVID-19, which has not been described to the same extent during MERS or SARS, nor has the cutaneous rash. Knowing the importance of MCs to lung inflammation and cutaneous rash, this strengthens the hypothesis that, although MCs could be involved in severe coronavirus disease caused by related viruses, their activation in COVID-19 disease may be especially severe and important for pathogenesis of this disease. However, MCs are also immune cells that are central to containing certain infections and their blockade can lead to enhanced infection in specific contexts. Accordingly, whether interventions that inhibit MCs or their products can successfully block COVID-19 in a way that prevents immune pathology and coagulopathy, without preventing infection clearance, is tested. An experiment to therapeutically intervene to reduce MC-induced inflammation during infection by SARS-CoV-2 in the mouse model (FIG. 3) is undertaken in order to establish the efficacy of this therapeutic strategy.

Having now generally described the invention, the same will be more readily understood through reference to the following examples, which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1: Efficacy of Drugs Targeting MCs or their Products

The efficacy of drugs targeting MCs or their products is tested at 2 doses of the coronavirus SARS-CoV-2, 1×10⁵ TCID₅₀ and either 5×10⁵ or 1×10⁶ TCID₅₀. Mice are inoculated intra-nasally in a 50 μL volume applied in 25 μL to each nostril containing virus in a phosphate-buffered solution to establish a lower respiratory tract infection. The K18-hACE2 mouse strain that has knocked-in human angiotensin-2 is used, which is known to be susceptible to SARS-CoV-2. Alternatively, or in addition, a group of C57BL/6 that express a transient hACE2 transgene can be used as an alternative model. This system is established by intranasal infection of wild-type mice with human ACE2 receptor using adenoviral-associated vectors (AAVs).

Controls are used both before starting the experiment with a few mice, by flow cytometry to show expression of human ACE2 receptor in various organs, and also internally during the experiment (e.g., use RNA for validation at the same time the RNA is used for virus detection) to validate the transgene expression.

Using the intranasal route of infection, daily injections of 10 mg/kg of TY-51469, or 10 mg/kg nafamostat mesylate, or Cromolyn (3 mg/mouse/day) or ketotifen (0.6 mg/mouse/day) or other drugs described in the application within their target dose ranges are used to investigate the effects of MC-stabilization or MC product inhibition. The mice in the control group are subjected to daily intraperitoneal injection of saline. Mice are observed for clinical scoring and the survival assessed daily. Targeting MCs with stabilizing compounds or MC-product targeting compounds is hypothesized to improve clinical scores and/or survival of the mice. At various time points, blood and tissue are collected from another group of mice. From blood, blood smears are assessed for coagulation, which is expected to be blocked by the therapeutic strategies described herein. Blood is also used to measure biomarkers of coagulation and inflammation such as MC proteases themselves e.g. chymase and tryptase, or other indicators of inflammation and coagulation such as CRP, fibrinogen-related products, complement activation products, cytokines and others using ELISA-based methods. These products are expected to be reduced during treatment of COVID-19 disease in animals at one or several time points, indicating the efficacy of the therapeutic regimens. Tissue collection is used to quantitate tissue damage (especially lung damage) by histology, and cellular read-outs such as cellular infiltration in the lung tissue. The therapeutic strategies described herein are expected to improve these read-outs of inflammation, which are correlated with severe disease, thus demonstrating therapeutic efficacy.

### Methods

RNA Isolation from Mouse and Primate Tissues and PCR-Based Quantification of SARS-CoV-2

Organs harvested from AAV9-hACE2 knocked-in mice and NHPs were transferred to Lysing Matrix Y tubes (MPBio, #116960050-CF) containing 0.5 mm diameter Yttria-Stabilized Zirconium Oxide beads. 500 μL 5% FBS DMEM was added into each tube and tissues were homogenized with a handheld homogenizer (MPBio SuperFastPrep-1) for 1 min. Total RNA was extracted from all samples using E.Z.N.A. Total RNA Kit I (Omega Bio-tek) according to the manufacturer's instructions and samples were analysed by real-time quantitative reverse transcription-PCR (RT-qPCR) for the detection of SARS-CoV-2 in mouse and NHP samples as previously described [Corman, V. M. et al., Euro Surveill 25: (2020); Lu, X. et al., Emerg Infect Dis 26: (2020)].

### Toluidine Blue Staining

For the toluidine blue staining protocol to identify mast cells (MCs), tissue sections were fixed in Carnoy's solution for 30 min at room temperature. Following fixation, sections were stained using 0.1% toluidine blue stain (Sigma-Aldrich, #198161) for 20 min and excess dye was removed by gently washing in running tap water followed by rinsing in distilled water. Sections were then dehydrated quickly in 95% alcohol followed by 2 changes in 100% alcohol and mounted using permanent mounting medium (VectaMount, #H-5000).

### Histology

Paraformaldehyde-fixed tissues were snap frozen in O.C.T compound (Tissue-Tek, Sakura) and sectioned to 15 μm thickness. For the toluidine blue staining protocol to identify MCs, sections were fixed in Carnoy's solution for 30 min at room temperature. Following fixation, sections were stained using 0.1% toluidine blue stain (Sigma-Aldrich, #198161) for 20 min and excess dye was removed by gently washing in running tap water followed by rinsing in distilled water. Sections were then dehydrated quickly in 95% alcohol followed by 2 changes in 100% alcohol and mounted using permanent mounting medium (VectaMount, #H-5000). For hematoxylin and eosin staining, air dried sections were rehydrated using a graded series of alcohol and stained using modified Harris hematoxylin (Sigma-Aldrich, #HHS32) for 10 min followed by a wash in tap water and two changes in distilled water. Sections were briefly dipped in 1% acid alcohol solution and quickly rinsed in distilled water before differentiating using 0.05% lithium carbonate solution for 1 minute. Sections were washed in distilled water and dehydrated using 95% alcohol followed by a counter stain using 0.25% eosin y (Sigma-Aldrich, #HT110232). Finally, sections were rinsed in 95% alcohol to remove excess eosin stain followed by 2 changes in 100% alcohol and air dried before mounted using permanent mounting medium (VectaMount, #H-5000). Images were obtained using a light microscope (Nikon) and processed using ImageJ Fiji software.

### Immunostaining of MCs

Tissue sections were permeabilized using 0.3% Triton X-100 in PBS for 30 min at room temperature followed by incubation with blocking buffer (0.1% Saponin+5% BSA in PBS) for 2 h at room temperature. Mast cells were probed using heparin binding Avidin conjugated to FITC (BD Pharmingen, #554057) for overnight at 4° C. Sections were washed 3-4 times using PBS before mounting using Fluoroshield mounting medium containing DAPI (Sigma-Aldrich, #F6057). Images were acquired using THUNDER Imaging Systems (Leica).

### Example 2: Infection of AAV-hACE2 Knock-In Mice with SARS-Cov-2

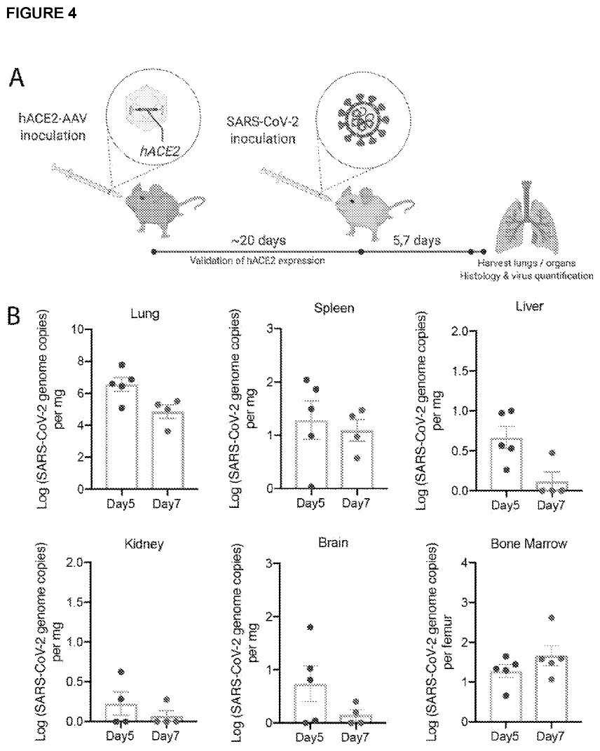

C57Bl/6 mice to be infected with AAV9-hACE2 were purchased from InVivos, Singapore, and housed in the Duke-NUS Vivarium prior to use. As shown in FIG. 4A, mice were anesthetized with xylazine/ketamine and inoculated intranasally with 0.5×10¹¹ PFU of AAV9-hACE2 delivered in 30 μL, alternating droplets between both nares. SARS-CoV-2 infections were performed 21 days later to allow maximal expression of hACE2. AAV9-hACE2 knock-in mice were transferred to the Duke-NUS ABSL3 facility for SARS-CoV-2 infections. Mice were anesthetized with isoflurane for nasal inoculations. They were infected with 6×10⁵ TCID₅₀/mL of SARS-CoV-2 isolate WX-56 via nasal inoculation (6 μL per nostril) and were subsequently weighed daily. Blood was collected on 1, 3, 5 and 7-days post-infection via cheek bleed. Mice were euthanized at 5 and 7 days post-infection and organs were harvested for RNA isolation and tissue sectioning. To isolate mouse serum, blood was allowed to clot at room temperature for 30 min prior to clarifying by centrifugation at 15,000 rpm in a table-top centrifuge. Mouse serum was inactivated by 30 minutes incubation at 56° C. to remove from the containment facility prior to further testing. Virus RNA quantification from the organs harvested from infected mice (FIG. 4B) shows detection in the lung, spleen, liver, kidney, brain, and bone marrow both Days 5 and 7.

### Mast Cell Degranulation in SARS-CoV-2 Infected Mice

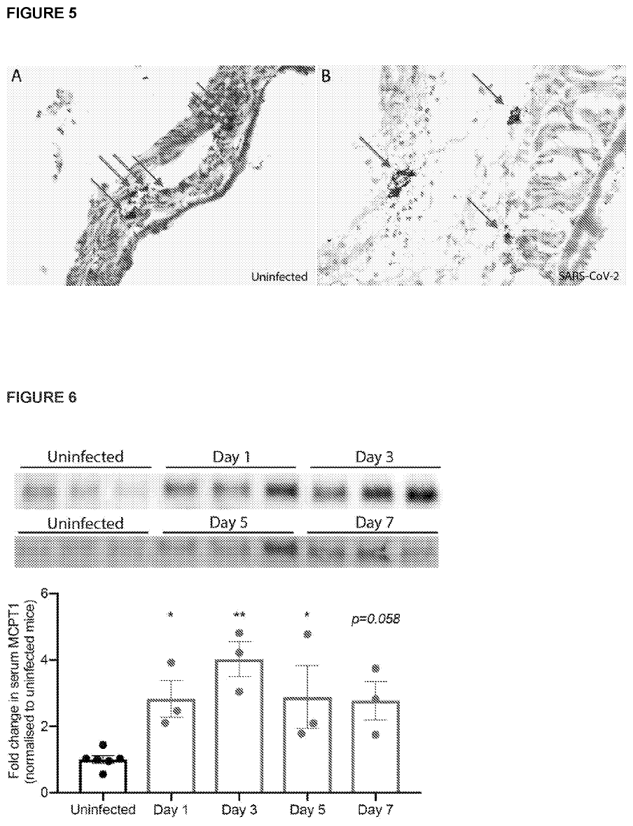

Histological images of toluidine blue-stained trachea sections from uninfected and SARS-CoV-2 infected mice (FIGS. 5A and 5B, respectively) show degranulation of mast cells (MCs) in SARS-CoV-2 infected mice. In uninfected tissues in panel A, arrows indicate MCs that could be identified by toluidine blue, all of which were resting and not degranulating. In panel B, degranulating MCs could be observed in SARS-CoV-2 infected mice (indicated by arrows) as well as tissue edema and airway narrowing.

### Detection of MCPT1 in Mouse Serum

Because mouse serum had been heat-inactivated, potentially denaturing proteins, we used a western blot to detect mast cell protease-1 (MCPT1), also known as beta-chymase, levels in the blood. Serum was diluted 1:10 in PBS and denatured in 2× laemmli buffer (Bio-Rad, #1610737) before serum proteins were fractionated by SDS-PAGE. Proteins were transferred onto PVDF membrane electrophoretically, which was blocked with 5% milk in TBST. Serum chymase was detected using Anti-Mast Cell Chymase antibody (Abcam, #ab2377,1:250) and Goat anti-Mouse IgG (H+L) Cross-Adsorbed Secondary Antibody, HRP (ThermoFisher Scientific, #G21040, 1:10000). Densitometric analysis was performed using Fiji software (ImageJ, NIH). MC-derived proteases were systemically elevated after infection. Western blot images after chymase detection in serum Days 3, 5 and 7 post- SARS-CoV-2 infection (FIG. 6), which was quantitated by densitometry from 3 individual mouse samples and presented as fold-increase over uninfected controls. Error bars represent the SEM. Chymase was significantly elevated in serum of infected mice compared to uninfected controls, determined by 1-way ANOVA with Dunnett's post-test; p<0.05, p<0.01.

### Example 3: SARS-CoV-2 Infection of Non-Human Primates (NHPs)

Cynomolgus macaques (Macaca fascicularis) were purchased from the SingHealth colony, free of antibodies against SARS-CoV-2. Infection studies were performed under BSL3 containment in the Duke-NUS Medical School ABSL3 facility. Prior to infection, NHPs were implanted with temperature transponders (Star-Oddi, Iceland). Body temperature was monitored every 15 min using a surgically implanted temperature sensor and rectally whenever the animals were anesthetized. For infection and sampling, animals were sedated with an intramuscular injection of ketamine (10-15 mg/kg) and medetomidine (0.05 mg/kg). Weight was recorded and a physical inspection was performed. Following initial sedation, 5% isoflurane was applied to achieve deeper anesthesia. A laryngoscope was used to intubate using an endotracheal (ET) tube. A 3 mL disposable luer lock syringe with 100 μL 3×10⁷ TCID₅₀/mL of SARS-CoV-2 isolate WX-56 was attached to ET tube connector for intratracheal infection. After injecting the virus, the ET tube was flushed with 1 mL of PBS to clear any residual inoculum. Animals were extubated and IV atipamezole was given to partially reverse medetomidine and facilitate faster recovery of the animals. Post-infection, animals were observed twice daily for activity and observation of clinical signs. At days 0, 1, 3 5, 7, 9, 14 and 21 post-infection, the animals were anesthetized and intubated using the same technique used for infection. Animals were weighed and blood samples collected from the femoral vein in CPT tubes. Nasal, rectal, throat and eye swabs were collected. Nasal rinse and lung lavage were performed with 500 μL and 6 ml PBS, respectively. The NHPs were euthanized at 21 days post-infection to allow for a full necropsy. Gross tissue observations were characterized by veterinarians and recorded upon necropsy. Blood, CSF, and tissues were harvested for RNA detection and histology.

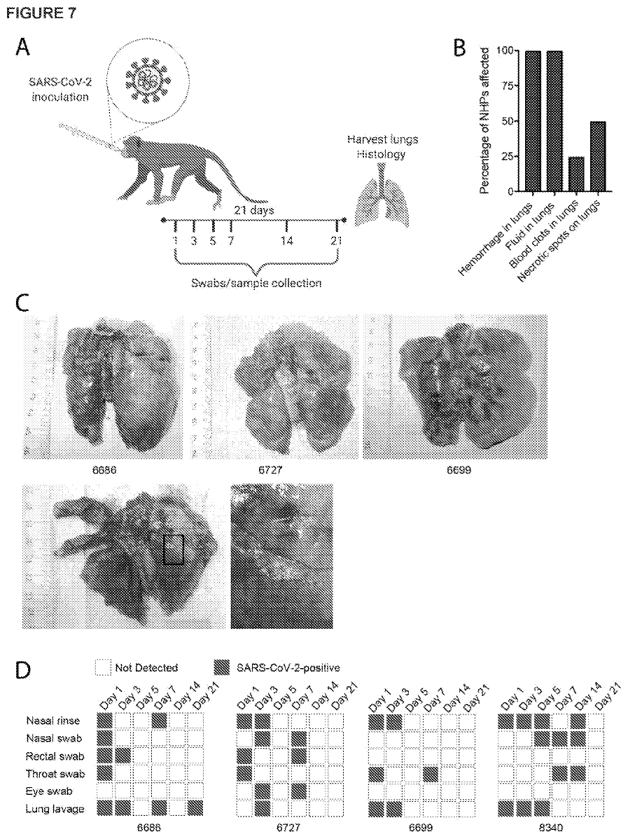

The lung pathology of Cynomolgus macaques infected with SARS-CoV-2 is shown in FIG. 7. Cynomolgus macaques were infected intra-tracheally with SARS-CoV-2 and monitored for 21 days prior to necropsy (FIG. 7A). Abnormal findings related to lung tissue observed at the time of necropsy were recorded and effected all animals, and included lung hemorrhage, fluid in the lungs, blood clots and necrotic spots (FIG. 7B). Images of NHP lungs showing areas of hemorrhaging and necrotic spots on the surface (boxed region is enlarged) are represented in FIG. 7C. PCR confirmed viral infection for multiple days in several mucosal tissues, lung lavage, and nasal rinses (FIG. 7D).

Widespread MC Activation Coincides with Lung Pathology

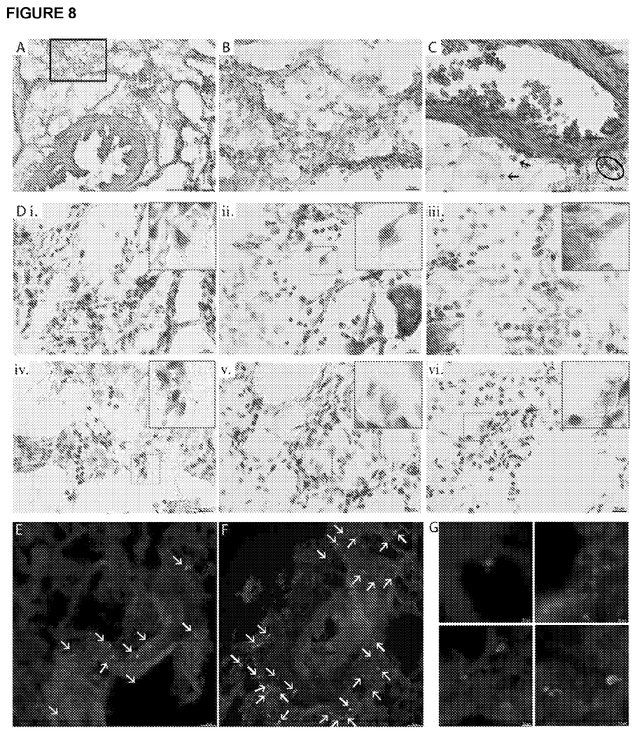

Lung tissue sections from Cynomolgus macaques infected with SARS-CoV-2 (described above) underwent histological assessment 21 days post-infection by H&E staining (FIG. 8A-D) or MC heparin and DAPI staining (FIG. 8E-G). H&E staining showed hemorrhaging of the tissue and free red blood cells (RBCs) within the lung alveolar spaces (FIG. 8A). An inset corresponding to the boxed region of panel A is shown in FIG. 8B. RBCs were observed in the tissue proximal to a blood vessel, indicated by arrows with cellular infiltrates circled, in FIG. 8C. Multiple examples of degranulating or hypogranulated MCs are provided, observed in toluidine blue stained lung tissue sections in FIG. 8D. The MCs are enlarged in the boxed insets. MCs from various regions of the lungs are presented, including i. lower lung tissue, ii. & iii. Trachea, iv. Lung tissue proximal to a bronchiole, and v. & vi. Lung tissue with hemorrhaging. Lung sections were stained for MC heparin to indicate the location of MC granules (light spots) and DAPI to identify cellular nuclei and tissue structures in MCs indicated by white arrows (FIG. 8E-G). MCs were observed degranulating in the lung of SARS-CoV-2 infected primates in sections of a biopsy of lung tissue that did not have overt hemorrhaging visible on the lung surface at necropsy (FIG. 8E). MCs appeared more densely packed in the lung biopsy from a hemorrhagic lobe of the lung and again, degranulation was observed based on staining for MC-heparin (FIG. 8F). Degranulating MCs are presented at higher magnification in FIG. 8G.

### Example 4: Analysis of Human Clinical Samples

### Microarray Analysis

The data associated with human transcriptional responses was approved by the SingHealth Combined Institutional Review Board (CIRB 2017/2374). The detailed study design and protocol has been described previously [Ong, E. Z., et al., EBioMedicine. 2021 March; 65: 103262. doi: 10.1016/j.ebiom.2021.103262. Epub 2021 Mar. 7], where whole blood transcript expression was measured in the severe and mild COVID-19 patients by the Affymetrix GeneChip Human Gene 2.0 ST Array. The raw data for the microarray profiling is available at Array Express (E-MTAB-9721), and the log 2 counts are generated by the Transcriptome Analysis Console (Thermo Fisher), analyzed between the different days relative to peak severity with regards to respiratory function (Day 0). Temporal gene expression was analyzed by EDGE software based on the log 2 intensity counts [Storey, J. D., Xiao, W., Leek, J. T., Tompkins, R. G. & Davis, R. W. Proc Natl Acad Sci USA 102: 12837-12842 (2005)], and genes that were significantly altered in the severe COVID-19 patients were identified based on p-value and q-value<0.05. The genes from the MC-specific and the MC/Basophil phenotype were obtained from [Dwyer, D. F., Barrett, N. A., Austen, K. F. & Immunological Genome Project, C. Nat Immunol 17: 878-887 (2016)], and Partek® Genomics Suite® software was used to tabulate the Least Square Means (LSMeans) values. Genes of increased expression during the acute phase or recovery phase were then further stratified. Normalized expression was tabulated by taking the average LSMeans values of all MC and MC/Basophil phenotype genes that were of increased expression during the acute phase. Heatmaps and graphs were constructed using Prism 9.0.2 software. For analysis of the microarray or nCounter datasets, Z-score transformation was performed as described previously [Cheadle C, et al., J Mol Diagn. 2003 May; 5(2): 73-81. doi: 10.1016/S1525-1578(10)60455-2]. To identify differentially expressed genes (DEGs) between symptomatic and asymptomatic subjects at baseline, Partek Genomics Suite Analysis v.7 software was used and Bonferroni's correction was performed based on the total number of 34,667 genes that were detected by microarray, using P<0.05. No cut-off on fold change was imposed. For pathway analysis, the identified DEGs were used as input data, and analyzed against the Reactome database using the Enrichr tool [Kuleshov M V, et al., Nucleic Acids Res. 2016 Jul. 8; 44(W1):W90-7. doi: 10.1093/nar/gkw377]. Both P values and combined scores for each enriched pathway were obtained from the Enrichr tool analysis using algorithms that are described in greater detail by Kuleshov et al., [Kuleshov M V, et al., Nucleic Acids Res. 2016 Jul. 8; 44(W1):W90-7. doi: 10.1093/nar/gkw377]. Volcano plots were constructed using Prism v.8.1.0 software. To evaluate whether there was any statistically significant difference in specific Reactome pathways between symptomatic and asymptomatic subjects, the average Z-scores of all genes in each of the pathway were plotted. An unpaired, Student's t-test was then used to assess the statistical significance of the observed differences. The ROC curves for the various pathways were also determined using the average Z-scores of all genes in the UPR, sumoylation and TCA cycle pathway and plotted using Prism v.8.1.0 software. Ingenuity software was used to generate gene network diagrams.

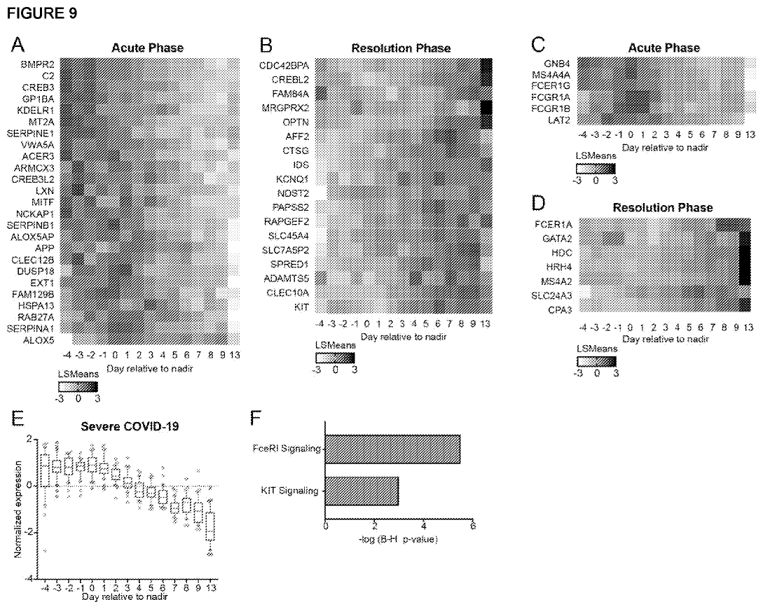

Transcriptional signatures of MC-associated genes with severe COVID-19 are shown in FIG. 9. Genes that were significantly regulated in severe COVID-19 patients and associated with an MC-specific phenotype are shown in FIG. 9A-B, whereas those associated with a MC/basophil phenotype are shown in FIG. 9C-D. A heatmap shows the LSmean expression values of MC-specific or MC/basophil phenotype genes in severe COVID-19 patients (n=6) at the various days relative to the peak severity with respect to respiratory function (day 0). Clusters of genes that were significantly upregulated during the acute phase (FIGS. 9A and C) or resolution phase (FIGS. 9B and D) are presented. FIG. 9E shows normalized expression levels of the MC-specific genes shown in A and C over time, in the severe COVID-19 patients. Pathway analysis indicates a significant perturbation of pathways associated with MC function and/or MC-precursor maturation (FIG. 9F).

### Chymase Detection in Clinical Samples

Patients with confirmed SARS-CoV-2 infection in Singapore were recruited in accordance with protocols approved by the institutional IRB, DSRB domain E, (#2020/00120) and informed consent was taken from all patients. Serum samples from acute patients, <7 days of illness, were tested for chymase using the Human mast cell chymase I (CMA-I) kit (BlueGene Biotech, catalogue number E01M0368), according to manufacturer's instructions. Chymase concentration values obtained and previously published using the same kit for healthy control and DENV patients from Singapore [St John, A. L., Rathore, A. P. S., Raghavan, B., Ng, M. L., Abraham, S. N. eLife (2013)] were compared to the values obtained in SARS-CoV-2 patients.

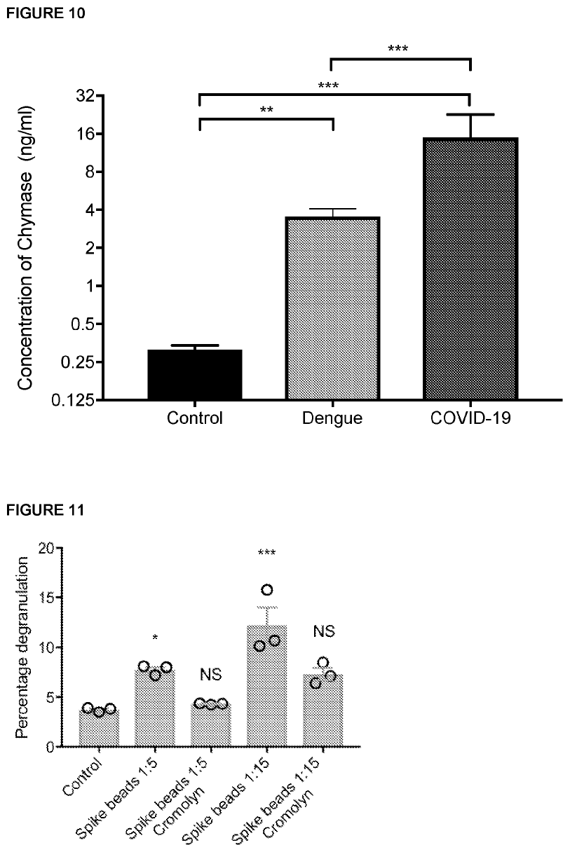

Levels of chymase in the serum of acute COVID-19 patients recruited in Singapore were compared to the concentrations previously detected and reported in a study of acute dengue patients [St John, A. L., Rathore, A. P. S., Raghavan, B., Ng, M. L., Abraham, S. N. eLife (2013)], and to healthy controls. FIG. 10 shows the serum chymase level in COVID19 patients to be about 50 times higher than in healthy controls and about 4 times higher than seen in the comparison Dengue patients. Concentrations were compared by 1-way ANOVA with Bonferroni's post-test to determine p-values. N=10 for controls, N=108 for dengue and N=3 for COVID-19. For **, p<0.01; for*** p<0.001.

### Example 5: Inhibition of Spike Protein-Induced MC Degranulation

### Mast Cell Stabilizer Inhibits SARS-CoV-2 Spike Protein-Induced MC Degranulation

Purified His-Tag SARS-CoV2 spike protein was obtained using a published protocol [Stadlbauer D, et al., Curr Protoc Microbiol. (2020) June; 57(1):e100. doi: 10.1002/cpmc.100] and was used to coat Polybead® carboxylate microspheres (Polysciences Inc., Cat #08226) as recommended by the manufacturer using 200 μg of protein. The purified recombinant spike protein was conjugated to beads to approximate the size of virus particles. A β-hexosaminidase assay was performed as previously described [St John, A. L. et al., Proc. Natl Acad. Sci. USA 108: 9190-9195 (2011)] with the following groups: bone marrow derived mast cells (BMMCs) obtain from C57Bl/6 mouse bone marrow+RPMI medium, BMMCs+ionomycin, BMMCs+SARS-CoV2 spike-coated beads ratio 1:5, BMMCs+SARS-CoV2 spike-coated beads ratio 1:15, BMMCs+SARS-CoV2 spike-coated beads ratio 1:5+Cromolyn (10 μM), and BMMCs+SARS-CoV2 spike-coated beads ratio 1:15+Cromolyn (10 μM). The absorbance at 405 nm was measured using a plate reader (Spark 10M, Tecan). Percentage degranulation was calculated by dividing the absorbance in supernatant with the sum of absorbance in both supernatant and cell lysate.

Results show that significant and dose-dependent mast cell degranulation was induced by spike-coated beads, but not in mast cells treated with the mast cell stabilizing drug cromolyn (FIG. 11).

### REFERENCES

Any listing or discussion of an apparently prior-published document in this specification should not necessarily be taken as an acknowledgement that such document is part of the state of the art or is common general knowledge.
1. Arentz, M. et al. Characteristics and Outcomes of 21 Critically Ill Patients With COVID-19 in Washington State. JAMA 323, 1612-1614 (2020).
2. Cheadle C, Vawter M P, Freed W J, Becker K G. Analysis of microarray data using Z score transformation. J Mol Diagn. 2003 May; 5(2): 73-81. doi: 10.1016/S1525-1578(10)60455-2.
3. Corman, V. M. et al., Detection of 2019 novel coronavirus (2019-nCoV) by real-time R T-PCR. Euro Surveill 25 (2020).
4. Corey, B. L., Mascola, J. R., Fauci, A. S. & Collins, F. S. A strategic approach to COVID-19 vaccine R&D. Science 368, 948-950 (2020).
5. Dolhnikoff, M. et al. Pathological evidence of pulmonary thrombotic phenomena in severe COVID-19. Journal of Thrombosis and Haemostasis 18, 1517-1519 (2020).
6. Dwyer, D. F., Barrett, N. A., Austen, K. F. & Immunological Genome Project, C. Expression profiling of constitutive mast cells reveals a unique identity within the immune system. Nat Immunol 17, 878-887 (2016).
7. Eastman, R. T. et al. Remdesivir: A Review of Its Discovery and Development Leading to Emergency Use Authorization for Treatment of COVID-19. ACS Cent Sci 6, 672-683 (2020).
8. Guan, W. J. et al. Comorbidity and its impact on 1590 patients with COVID-19 in China: a nationwide analysis. Eur Respir J 55, 2000547 (2020).
9. Hsieh, J. T., Rathore, A. P. S., Soundarajan, G. & St John, A. L. Japanese encephalitis virus neuropenetrance is driven by mast cell chymase. Nat Commun 10, 706 (2019).
10. https://www.fda.gov/emergency-preparedness-and-response/mcm-legal-regulatory-and-policy-framework/emergency-use-authorization #covidtherapeutics. (2020).
11. https://www.niaid.nih.gov/news-events/nih-clinical-trial-shows-remdesivir-accelerates-recovery-advanced-covid-19. (2020).
12. https://www.who.int/blueprint/priority-diseases/key-action/novel-coronavirus-landscape-ncov.pdf. (2020).
13. Kimmig, L. M. et al. IL6 inhibition in critically ill COVID-19 patients is associated with increased secondary infections. medRxiv, 2020.2005.2015.20103531 (2020).
14. Kong, W. & Agarwal, P. P. Chest Imaging Appearance of COVID-19 Infection. Radiology: Cardiothoracic Imaging 2, e200028 (2020).
15. Kuleshov M V, Jones M R, Rouillard A D, Fernandez N F, Duan Q, Wang Z, Koplev S, Jenkins S L, Jagodnik K M, Lachmann A, McDermott M G, Monteiro C D, Gundersen G W, Ma'ayan A. Enrichr: a comprehensive gene set enrichment analysis web server 2016 update. Nucleic Acids Res. 2016 Jul. 8; 44(W1):W90-7. doi: 10.1093/nar/gkw377.
16. Landa, N., Mendieta-Eckert, M., Fonda-Pascual, P. & Aguirre, T. Chilblain-like lesions on feet and hands during the COVID-19 Pandemic. International Journal of Dermatology 59, 739-743 (2020).
17. Lu, X. et al., U S CDC Real-Time Reverse Transcription PCR Panel for Detection of Severe Acute Respiratory Syndrome Coronavirus 2. Emerg Infect Dis 26: (2020).
18. Masri, M. F. B., Mantri, C. K., Rathore, A. P. S. & John, A. L. S. Peripheral serotonin causes dengue virus-induced thrombocytopenia through 5HT2 receptors. Blood 133, 2325-2337 (2019).
19. Morassi, M. et al. Stroke in patients with SARS-CoV-2 infection: case series. Journal of Neurology 267, 2185-2192 (2020).
20. Mulangu, S. et al. A Randomized, Controlled Trial of Ebola Virus Disease Therapeutics. N Engl J Med 381, 2293-2303 (2019).
21. Ong E Z, Kalimuddin S, Chia W C, Ooi S H, Koh C W, Tan H C, Zhang S L, Low J G, Ooi E E, Chan K R. Temporal dynamics of the host molecular responses underlying severe COVID-19 progression and disease resolution. EBioMedicine. 2021 March; 65: 103262. doi: 10.1016/j.ebiom.2021.103262. Epub 2021 Mar. 7.
22. Organization, W. H. WHO Timeline—COVID-19. 2020 [cited 2020 Jun. 14] Available from: https://www.who.int/news-room/detail/27-04-2020-who-timeline---covid-19.
23. Pinto, D. et al. Cross-neutralization of SARS-CoV-2 by a human monoclonal SARS-CoV antibody. Nature 583, 290-295 (2020).
24. Rathore, A. P. et al. Dengue virus-elicited tryptase induces endothelial permeability and shock. J Clin Invest 130, 4180-4193 (2019).
25. Riphagen, S., Gomez, X., Gonzalez-Martinez, C., Wilkinson, N. & Theocharis, P. Hyperinflammatory shock in children during COVID-19 pandemic. The Lancet 395, 1607-1608 (2020).
26. Stadlbauer D, Amanat F, Chromikova V, Jiang K, Strohmeier S, Arunkumar G A, Tan J, Bhavsar D, Capuano C, Kirkpatrick E, Meade P, Brito R N, Teo C, McMahon M, Simon V, Krammer F. SARS-CoV-2 Seroconversion in Humans: A Detailed Protocol for a Serological Assay, Antigen Production, and Test Setup. Curr Protoc Microbiol. 2020 June; 57(1):e100. doi: 10.1002/cpmc.100. PMID: 32302069; PMCID: PMC7235504.
27. St John, A. L. et al., Immune surveillance by mast cells during dengue infection promotes natural killer (N K) and NKT-cell recruitment and viral clearance. Proc. Natl Acad. Sci. USA 108, 9190-9195 (2011).
28. St John, A. L., Rathore, A. P., Raghavan, B., Ng, M. L. & Abraham, S. N. Contributions of mast cells and vasoactive products, leukotrienes and chymase, to dengue virus-induced vascular leakage. Elife 2, e00481 (2013).
29. St John, A. L. & Rathore, A. P. S. Early Insights into Immune Responses during COVID-19. J Immunol 205, 555-564 (2020).
30. Storey, J. D., Xiao, W., Leek, J. T., Tompkins, R. G. & Davis, R. W. Significance analysis of time course microarray experiments. Proc Natl Acad Sci USA 102, 12837-12842 (2005).
31. Tchougounova, E., Pejler, G. & Abrink, M. The chymase, mouse mast cell protease 4, constitutes the major chymotrypsin-like activity in peritoneum and ear tissue. A role for mouse mast cell protease 4 in thrombin regulation and fibronectin turnover. J Exp Med 198, 423-431 (2003).
32. Thanh Le, T. et al. The COVID-19 vaccine development landscape. Nat Rev Drug Discov 19, 305-306 (2020).
33. Tissera, H. et al. Chymase Level Is a Predictive Biomarker of Dengue Hemorrhagic Fever in Pediatric and Adult Patients. J Infect Dis 216, 1112-1121 (2017).
34. Wang, C. et al. A human monoclonal antibody blocking SARS-CoV-2 infection. Nat Commun 11, 2251 (2020).
35. Wang, M. et al. Remdesivir and chloroquine effectively inhibit the recently emerged novel coronavirus (2019-nCoV) in vitro. Cell Res 30, 269-271 (2020).

1.-14. (canceled)

15. A method of prophylaxis or treatment of a coronavirus-induced disease, wherein the disease is characterized by mast cell degranulation, acute inflammation, pulmonary pathology and/or vascular pathology, comprising administering to a subject in need thereof an efficacious amount of a composition comprising a mast cell stabilizer and/or inhibitor of mast cell products.

16. The method of claim 15, wherein the coronavirus is selected from the group comprising SARS-CoV, SARS-Cov-2 and MERS-CoV.

17. The method of claim 15, wherein the mast cell stabilizer is selected from one or more of a group consisting of cromolyn, nedocromil, pemirolast, lodoxamide, tranilast, glucosamine, N-acetylglucosamine, FPL 52694, aloe vera, quercetin, chondroitin sulfate, dehydroleucodine, mast cell stabilizer TF002, rupatadine, loratadine, cetirizine, clemastime, fexofenadine, diphenhydramine, chlorpheniramine, azelastine, olopatadine, naphazoline, ketotifen, emedastine, ebrotidine, calcium channel blocker, a cytochrome P450 inhibitor, a histamine antagonist,
and the inhibitor of mast cell products is selected from one or more of a group consisting of zafirlukast, ketanserin, montelukast, pranlukast, zileuton, SM-12502, rupatadine, PAF-targeting antibodies, xanthine derivatives, methylxanthines like theophylline oxtriphylline, dyphylline, aminophylline, bupropion, curcumin, catechins, aprotinin, serpin, a chymase inhibitor, TY-51469, chymostatin, leupeptin, APC-336, SUN-C8257, NK3201, R0566852, BCEAB, NK3201, TEI-E548, APC-2095, RWJ-355871, TPC-806, ZIGPFM, AAPF-S-Bzl, Bowman-Birk soybean protease inhibitor, BI-1942, TEI-f00806, BAY-1142524, fulacimstat, ASB17061, Polygonum, SFTI-1 and derivatives, bevacizumab, ranibizumab, lapatinib, sunitinib sorafenib, axitinib, pazopanib, thiazolidinediones, benzoxazole, benzthiazole, benzinidzole, CP105,696, laropiprant, acetylsalicylic acid (ASA), indomethacin, sodium meclofenamate (FEN), phenylbutazone (PB), phloretin phosphates (PP), SC-19220, diethylcarbamazine citrate (DECC), protamine and polybrene, a tryptase inhibitor, nafamostat mesylate, BMS-262084, BMS-363131, BSM-36130, Guanadino β-lactams that inhibit tryptase, delta inhibitors of tryptase, benzamidine dimers that inhibit tryptase, piperidine containing 4-carboxy azetidinone tryptase inhibitors, APC-2059, BAY 443428, phenylglycylcarbonyl benzylamines, Peptidyl heterocyclic ketones, Guanidino Bicyclic lactam, Amino or Amidino dimers, Peptidomimetic inhibitors, MOL-6131, RWJ-56423, RWJ-58643, RWJ-51084, BABIM (bis-(5-amidino-2-benzimidazoyl) methane, APD-8, AMG-126737, 4-chlorobenzyoyl ester of 4-hydroxytetronic acid and its p-toluate tetronic acid derivatives, M-58538, AY-0068, PMD-3027, Cyclotheonamide E4 and E5, and amidinobenzofuran derivatives.

18. The method of claim 15, comprising one or more mast cell stabilizer and/or inhibitor of mast cell products selected from the group consisting of ketotifen [IUPAC Name: 2-(1-methylpiperidin-4-ylidene)-6-thiatricyclo[8.4.0.03,7]tetradeca-1(14),3(7),4,10,12-pentaen-8-one], cromolyn [IUPAC Name: 5-[3-(2-carboxy-4-oxochromen-5-yl)oxy-2-hydroxypropoxy]-4-oxochromene-2-carboxylic acid], nafamostat mesylate [IUPAC Name: (6-carbamimidoylnaphthalen-2-yl) 4-(diaminomethylideneamino)benzoate; methanesulfonic acid], TY-51469 [IUPAC Name 2-[4-[(5-fluoro-3-methyl-1-benzothiophen-2-yl)sulfonylamino]-3-methylsulfonylphenyl]-1,3-thiazole-4-carboxylic acid] and ketanserin [IUPAC Name: 3-[2-[4-(4-fluorobenzoyl)piperidin-1-yl]ethyl]-1H-quinazoline-2,4-dione].

19. The method of claim 15, wherein the subject is administered:
cromolyn at 200 mg or more 4× a day to adults, or at 100 mg or more 4× a day to children;
ketotifen at 1 mg or more per day to adults or at 0.110 mg or more per pound of body weight to adults or children;
nafamostat at 20-50 mg or more by infusion, or 2-10 mg/kg or more per day;
TY-51469 at 0.1-100 mg/kg or more per day; and/or
Ketanserin at 10-100 mg or more twice a day.

20. A method of monitoring the efficacy of the method of prophylaxis or treatment of claim 15, comprising serially measuring the level and/or activity of a mast cell protease, preferably selected from the group consisting of chymase and tryptase, in at least one sample from said subject.

21. The method of claim 20, wherein the method of prophylaxis or treatment is according to claim 16.

22. The method of claim 20, wherein the method of prophylaxis or treatment is according to claim 17.

23. The method of claim 20, wherein the method of prophylaxis or treatment is according to claim 18.

24. The method of claim 20, wherein the method of prophylaxis or treatment is according to claim 19.

25. A method of diagnosing a subject as having a coronavirus-induced disease, wherein the disease is characterized by mast cell degranulation, acute inflammation, pulmonary pathology, vascular pathology, the method comprising:
(a) obtaining a biological sample from the subject;
(b) determining the level and/or activity of at least one biomarker in the biological sample from the subject;
(c) comparing the level and/or activity of the at least one biomarker in the biological sample to a reference level and/or activity of the at least one biomarker;
(d) identifying the subject as having the disease or having an increased risk of developing the disease if the level and/or activity of the at least one biomarker is greater than the reference level and/or activity of the at least one biomarker;
(e) preventing or treating the disease by administering an efficacious amount of a composition comprising a mast cell stabilizer and/or inhibitor of mast cell products.

26. The method of claim 25, wherein the mast cell stabilizer is selected from one or more of a group consisting of cromolyn, nedocromil, pemirolast, lodoxamide, tranilast, glucosamine, N-acetylglucosamine, FPL 52694, aloe vera, quercetin, chondroitin sulfate, dehydroleucodine, mast cell stabilizer TF002, rupatadine, loratadine, cetirizine, clemastime, fexofenadine, diphenhydramine, chlorpheniramine, azelastine, olopatadine, naphazoline, ketotifen, emedastine, ebrotidine, calcium channel blocker, a cytochrome P450 inhibitor, a histamine antagonist,
and the inhibitor of mast cell products is selected from one or more of a group consisting of zafirlukast, ketanserin, montelukast, pranlukast, zileuton, SM-12502, rupatadine, PAF-targeting antibodies, xanthine derivatives, methylxanthines like theophylline oxtriphylline, dyphylline, aminophylline, bupropion, curcumin, catechins, aprotinin, serpin, a chymase inhibitor, TY-51469, chymostatin, leupeptin, APC-336, SUN-C8257, NK3201, R0566852, BCEAB, NK3201, TEI-E548, APC-2095, RWJ-355871, TPC-806, ZIGPFM, AAPF-S-Bzl, Bowman-Birk soybean protease inhibitor, BI-1942, TEI-f00806, BAY-1142524, fulacimstat, ASB17061, Polygonum, SFTI-1 and derivatives, bevacizumab, ranibizumab, lapatinib, sunitinib sorafenib, axitinib, pazopanib, thiazolidinediones, benzoxazole, benzthiazole, benzinidzole, CP105,696, laropiprant, acetylsalicylic acid (ASA), indomethacin, sodium meclofenamate (FEN), phenylbutazone (PB), phloretin phosphates (PP), SC-19220, diethylcarbamazine citrate (DECC), protamine and polybrene, a tryptase inhibitor, nafamostat mesylate, BMS-262084, BMS-363131, BSM-36130, Guanadino β-lactams that inhibit tryptase, delta inhibitors of tryptase, benzamidine dimers that inhibit tryptase, piperidine containing 4-carboxy azetidinone tryptase inhibitors, APC-2059, BAY 443428, phenylglycylcarbonyl benzylamines, Peptidyl heterocyclic ketones, Guanidino Bicyclic lactam, Amino or Amidino dimers, Peptidomimetic inhibitors, MOL-6131, RWJ-56423, RWJ-58643, RWJ-51084, BABIM (bis-(5-amidino-2-benzimidazoyl) methane, APD-8, AMG-126737, 4-chlorobenzyoyl ester of 4-hydroxytetronic acid and its p-toluate tetronic acid derivatives, M-58538, AY-0068, PMD-3027, Cyclotheonamide E4 and E5, and amidinobenzofuran derivatives.

27. The method of claim 25, wherein the composition comprises one or more mast cell stabilizer and/or inhibitor of mast cell products selected from the group consisting of ketotifen [IUPAC Name: 2-(1-methylpiperidin-4-ylidene)-6-thiatricyclo[8.4.0.03,7]tetradeca-1(14),3(7),4,10,12-pentaen-8-one], cromolyn [IUPAC Name: 5-[3-(2-carboxy-4-oxochromen-5-yl)oxy-2-hydroxypropoxy]-4-oxochromene-2-carboxylic acid], nafamostat mesylate [IUPAC Name: (6-carbamimidoylnaphthalen-2-yl) 4-(diaminomethylideneamino)benzoate; methanesulfonic acid], TY-51469 [IUPAC Name 2-[4-[(5-fluoro-3-methyl-1-benzothiophen-2-yl)sulfonylamino]-3-methylsulfonylphenyl]-1,3-thiazole-4-carboxylic acid] and ketanserin [IUPAC Name: 3-[2-[4-(4-fluorobenzoyl)piperidin-1-yl]ethyl]-1H-quinazoline-2,4-dione].

28. The method of claim 25, wherein the subject is administered:
cromolyn at 200 mg or more 4× a day to adults, or at 100 mg or more 4× a day to children;
ketotifen at 1 mg or more per day to adults or at 0.110 mg or more per pound of body weight to adults or children;
nafamostat at 20-50 mg or more by infusion, or 2-10 mg/kg or more per day;
TY-51469 at 0.1-100 mg/kg or more per day; and/or
Ketanserin at 10-100 mg or more twice a day.

29. The method of claim 25, wherein the control sample is a sample from a healthy patient, a patient having a mild form of the coronavirus-induced disease, or a patient having a severe form of the coronavirus-induced disease.

30. The method of claim 25, wherein the biomarker is a mast cell protease, preferably selected from the group comprising chymase and tryptase.

31. The method of claim 30, wherein:
a) a mast cell protease level and/or activity greater than 1 standard deviation above the mean for patients during acute disease or during disease resolution indicates the subject should be monitored for severe disease and/or complications; or
b) a mast cell protease level above a normal level of about 300 pg/mL for tryptase or 3 ng/mL for chymase indicates the subject should be monitored for severe disease.

32. A composition comprising a mast cell stabilizer and/or inhibitor of mast cell products for prophylaxis or treatment of coronavirus-induced disease, wherein the disease is characterized by mast cell degranulation, acute inflammation, pulmonary pathology and/or vascular pathology.

33. The composition of claim 6, wherein the coronavirus is selected from the group consisting of Severe Acute Respiratory Syndrome-associated coronavirus (SARS-CoV), SARS-CoV-2 and Middle East Respiratory Syndrome-associated coronavirus (MERS-CoV).

34. The composition of claim 32, wherein the mast cell stabilizer is selected from one or more of a group consisting of cromolyn, nedocromil, pemirolast, lodoxamide, tranilast, glucosamine, N-acetylglucosamine, FPL 52694, aloe vera, quercetin, chondroitin sulfate, dehydroleucodine, mast cell stabilizer TF002, rupatadine, loratadine, cetirizine, clemastime, fexofenadine, diphenhydramine, chlorpheniramine, azelastine, olopatadine, naphazoline, ketotifen, emedastine, ebrotidine, calcium channel blocker, a cytochrome P450 inhibitor, a histamine antagonist,
and the inhibitor of mast cell products is selected from one or more of a group consisting of zafirlukast, ketanserin, montelukast, pranlukast, zileuton, SM-12502, rupatadine, PAF-targeting antibodies, xanthine derivatives, methylxanthines like theophylline oxtriphylline, dyphylline, aminophylline, bupropion, curcumin, catechins, aprotinin, serpin, a chymase inhibitor, TY-51469, chymostatin, leupeptin, APC-336, SUN-C8257, NK3201, R0566852, BCEAB, NK3201, TEI-E548, APC-2095, RWJ-355871, TPC-806, ZIGPFM, AAPF-S-Bzl, Bowman-Birk soybean protease inhibitor, BI-1942, TEI-f00806, BAY-1142524, fulacimstat, ASB17061, Polygonum, SFTI-1 and derivatives, bevacizumab, ranibizumab, lapatinib, sunitinib sorafenib, axitinib, pazopanib, thiazolidinediones, benzoxazole, benzthiazole, benzinidzole, CP105,696, laropiprant, acetylsalicylic acid (ASA), indomethacin, sodium meclofenamate (FEN), phenylbutazone (PB), phloretin phosphates (PP), SC-19220, diethylcarbamazine citrate (DECC), protamine and polybrene, a tryptase inhibitor, nafamostat mesylate, BMS-262084, BMS-363131, BSM-36130, Guanadino β-lactams that inhibit tryptase, delta inhibitors of tryptase, benzamidine dimers that inhibit tryptase, piperidine containing 4-carboxy azetidinone tryptase inhibitors, APC-2059, BAY 443428, phenylglycylcarbonyl benzylamines, Peptidyl heterocyclic ketones, Guanidino Bicyclic lactam, Amino or Amidino dimers, Peptidomimetic inhibitors, MOL-6131, RWJ-56423, RWJ-58643, RWJ-51084, BABIM (bis-(5-amidino-2-benzimidazoyl) methane, APD-8, AMG-126737, 4-chlorobenzyoyl ester of 4-hydroxytetronic acid and its p-toluate tetronic acid derivatives, M-58538, AY-0068, PMD-3027, Cyclotheonamide E4 and E5, and amidinobenzofuran derivatives.

35. The composition of claim 32, comprising one or more mast cell stabilizer and/or inhibitor of mast cell products selected from the group consisting of ketotifen [IUPAC Name: 2-(1-methylpiperidin-4-ylidene)-6-thiatricyclo[8.4.0.03,7]tetradeca-1(14),3(7),4,10,12-pentaen-8-one], cromolyn [IUPAC Name: 5-[3-(2-carboxy-4-oxochromen-5-yl)oxy-2-hydroxypropoxy]-4-oxochromene-2-carboxylic acid], nafamostat mesylate [IUPAC Name: (6-carbamimidoylnaphthalen-2-yl) 4-(diaminomethylideneamino)benzoate; methanesulfonic acid], TY-51469 [IUPAC Name 2-[4-[(5-fluoro-3-methyl-1-benzothiophen-2-yl)sulfonylamino]-3-methylsulfonylphenyl]-1,3-thiazole-4-carboxylic acid] and ketanserin [IUPAC Name: 3-[2-[4-(4-fluorobenzoyl)piperidin-1-yl]ethyl]-1H-quinazoline-2,4-dione].

36. The composition of claim 35, wherein;
cromolyn is formulated for administration to adults at 200 mg or more 4× a day, and children at 100 mg or more 4× a day;
ketotifen is formulated for administration orally at 1 mg or more per day in adults or 0.110 mg or more per pound of body weight including for children;
nafamostat mesylate is formulated for administration at 20-50 mg or more infusion, or 2-10 mg/kg or more per day;
TY-51469 is formulated for administration at 0.1-100 mg/kg or more per day; and/or
Ketanserin is formulated for administration at 10-100 mg or more twice a day.
